SEQUENCE LISTING (8 sequences)

SEQ ID NO: 1 - DNA, 20 bp, Artificial Sequence
  Forward primer for amplifying procollagen type I
ctcgaggtggacaccaccct

SEQ ID NO: 2 - DNA, 20 bp, Artificial Sequence
  Reverse primer for amplifying procollagen type I
cagctggatggccacatcgg

SEQ ID NO: 3 - DNA, 24 bp, Artificial Sequence
  Forward primer for amplifying MMP-1
attctactgatatcggggctttga

SEQ ID NO: 4 - DNA, 23 bp, Artificial Sequence
  Reverse primer for amplifying MMP-1
atgtccttggggtatccgtgtag

SEQ ID NO: 5 - DNA, 20 bp, Artificial Sequence
  Forward primer for amplifying fibronectin
aggttcgggaagaggttgtt

SEQ ID NO: 6 - DNA, 20 bp, Artificial Sequence
  Reverse primer for amplifying fibronectin
tggcaccgagatattccttc

SEQ ID NO: 7 - DNA, 24 bp, Artificial Sequence
  Forward primer for amplifying GAPDH
ccacccatggcaaattccatggca

SEQ ID NO: 8 - DNA, 24 bp, Artificial Sequence
  Reverse primer for amplifying GAPDH
tctagacggcaggtcaggtccacc

(19) United States
(12) Patent Application Publication
(10) Pub. No.: US 2009/0209020 A1
(43) Pub. Date: Aug. 20, 2009

(54) collagenous matrix with improved porosity and tensile strength and preparation method therefore by using mechanical stimulation system

(76) Inventors: Kyoung-Chan Park, Seoul (KR); Sun-Bang Kwon, Daejeon (KR); Hye-Ryung Choi, Seoul (KR)

Correspondence Address: THE NATH LAW GROUP, 112 South West Street, Alexandria, VA, 22314, US

(21) Appl. No.: 12/084,570

(22) PCT Filed: Nov. 6, 2006
(86) PCT No.: PCT/KR2006/004604

§ 371 Date: Jul. 17, 2008

(30) Foreign Application Priority Data
Nov. 7, 2005 (KR) 10-2005-0105929

(51) Int. Cl. C12N 11/04

(52) U.S. Cl. 435/182; 435/177

(57) ABSTRACT

The present invention relates to a method of preparing a collagen matrix with increased porosity and tensile strength by using mechanical stimulation system. More specifically, in the present invention, the cell-populated gel is being cultured under the condition that the physical forces is loaded for causing the matrix to move periodically and discontinuously. Resulting collagen matrix can be used for preparing an artificial skin or organs. Furthermore, this collagen matrix can be used as fillers for esthetic or therapeutic purposes.

### BACKGROUND OF THE INVENTION

1. Field of the Invention

The present invention relates to a method of preparing a collagen matrix with increased porosity and tensile strength by using mechanical stimulation system.

Especially in the present invention, the cells-containing gel is being stimulated under the condition that the physical force is loaded to a bottom of the collagen gel periodically and discontinuously. Physical force was transversely loaded to the collagen gel, thereby inducing the cells and collagen matrix to receive the different kinds of forces simultaneously. The induction transmits complex signals to the cell, and thus controlling the production and digestion of the collagen to produce the collagen matrix with increased porosity and tensile strength. The improved collagen matrix can be used for preparing an artificial organ and also as dermal fillers or substitutes.

2. Description of the Related Art

In particular, the biomaterials such as collagens, are used more and more in the pharmaceutical field, and are applied for reconstruction of damaged connective tissue and gene therapy. Collagen matrix is suitable for the various kinds of cell growth, and can be widely used for preparing the reconstructed tissues in a shape of matrix, gel or membrane, etc. However, collagen matrix, which can be prepared by gelling of collagen solution, is very weak and is not ideal for the culture of artificial organs such as artificial skin, cartilage, and bones so on. To solve the problems, many techniques such as usages of polymers including nylon, collagen mesh, and a mixture of collagen and chitosan were developed, but the obtained products were not satisfactory. In this point, a method to make more compact collagen matrix is very urgent and important for tissue engineering purposes.

An organism breathes, moves, and interacts with an environment continuously. However in experimental condition, the cells are cultured in the reactor under the controlled condition without any external stimulation. In other words, the culturing conditions such as the atmosphere, temperature, humidity, and etc. are totally different from the natural conditions of the cells. Thus, physical stimulation began to be used to provide more in-vivo like situation in culture systems.

It is reported that different mechanical stimuli provoke different cell responses. In osteocytes, shear stress induces the translocation of connexin43 to the membrane surface and that unopposed hemichannels formed by connexin43 serve as a novel portal for the release of PGE2 in response to mechanical strain (Cherian PP et al, Mol Biol Cell, 2005 16(7):3100-6). In addition, it is also reported that long-term intermittent compressive stimulation can increase collagen synthesis and mechanical properties of tissue-engineered cartilage (Waldman S D et al, Tissue Eng. September-October; 10(9-10): 1323-31, 2004). Based on these results, mechanical stimulations include a method of stretching (Wang J H et al, Ann Biomed Eng., 2005 33(3): 337-42; Katsumi A et al, J Biol chem., 2005 280(17): 16546-9), and of hydrostatic fluid pressure (HRP) into the cell culture solution (Mizuno et al, J Cell Physiol, 2002 193(3): 319-27) have been used for the culturing of bone and cartilage.

However, the effects of mechanical stimulation on dermal fibroblasts are not studied well. In literature, mechanical signals can regulate extracellular matrix gene expression of fibroblasts. Although action mechanism of mechanical stimulation is unknown, it is inferred that contraction of muscle or gravity is transmitted to the cytoskeleton through the extracellular matrix, and then is changed into intracellular signal (Sarasa-Renedo A, et al., Scand J Med Sci Sports, August; 15(4):223-230, 2005).

### SUMMARY OF THE INVENTION

In this invention, the present inventors tried to establish a method, in which the inventors can culture collagen matrix with good porosity and tensile strength. For tissue engineering technology, collagen matrix with good porosity and tensile strength is urgently needed. In this invention, the inventors used “transverse impulse loading” for the culturing of collagen matrix and found that “transverse impulse loading” dramatically increased collagen synthesis and collagen matrix remodeling. As a result, “transverse impulse loading” can produce collagen matrix with good porosity and tensile strength.

In one embodiment, the present invention provides a method of preparing a collagen matrix including mammalian cells, which comprise the steps of:

a) preparing a gel containing the mammalian cells by mixing the mammalian cells and a solution including a material selected from the group consisting of collagen, fibrin and a mixture thereof; and

b) stimulating the gel under the condition that physical force is loaded to the collagen gel periodically and discontinuously in order to make the collagen matrix with good porosity and tensile strength. The physical force is loaded to a bottom of the gels in order to increase the collagen production by the mammalian cells. The physical force is a transverse impulse loaded to the plane of collagen gels at one or more sites thereof. The gel is fixed at a peripheral side thereof and then is loaded by physical force at least one site of bottom of the collagen gel including the central site thereof. The stimulation is loaded at least two sites of the bottom of the collagen gel independently. The stimulation strength is 1.0×10⁻⁷ to 1.0×10⁻¹N/m² and the stimulation frequency is 0.01 to 500 cycles per a minute. The stimulation is generated by at least a cam attached to the cam-shaft which rotates at a speed of 0.01 to 500 cycles per a minute.

Step b) is performed by stimulating the gels containing the cells in a culture vessel made form elastic material which is polyethylene, polypropylene, ethylene-propylene copolymer, or silicon.

The cells are mixed at a concentration of 1×10³ to 1×10⁷ cells per 1 ml of the solution. The collagen is at least one selected from the group consisting of Type I collagen, Type III collagen, and Type IV collagen.

In another embodiment, the present invention provides a collagen matrix with increased porosity and tensile strength prepared by the production method as above. The present invention provides a collagen matrix having pores with a diameter of 0.1 to 100 μm, a porosity of 10% to 90%, and a tensile strength of 1 N/cm² to 200 N/cm².

In third embodiment, the present invention provides an artificial skin or artificial organ used for scaffold for culturing of artificial skin or organs comprising the collagen matrix being prepared according to the method of present invention. Still another object of the present invention is to provide a method of culturing an artificial skin or artificial organs by using the improved collagen matrix. The present invention provides a collagen scaffold used for culturing of artificial skin or organs, which comprises the collagen matrix being prepared according to the method of the present invention.

In fourth embodiment, the present invention provides fillers used for esthetic or therapeutic purposes which comprise the collagen matrix being prepared according to the method of present invention.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawing.

FIG. 1 is a schematic view of the mechanical stimulator.

FIG. 2 shows a representative cycle of displacement after physical conditioning according to Example 1-1.

FIG. 3 represents the pictures of artificial skin cultured on the matrixes of control model and stimulated model according to Example 2.

FIG. 4 shows increased dry weight of collagen gels cultured with mechanical stimulation according to Experimental Example 1.

FIG. 5 shows increased porosity of collagen gels cultured with mechanical stimulation according to Experimental Example 2.

FIG. 6 shows increased tensile strength of collagen gels cultured with mechanical stimulation according to Experimental Example 3.

FIG. 7A shows increased mRNA expression of collagen type I, MMP-1, and fibronectin in the collagen gels cultured under the mechanical stimulation, and FIG. 7B shows increased protein levels of collagen type I, TIMP-1, and TIMP-2 in the collagen gels cultured with mechanical stimulation according to Experimental Example 4.

### DETAILED DESCRIPTION OF THE ILLUSTRATED EMBODIMENTS

The collagen is the major extra-cellular matrix protein produced by fibroblast. Collagen constitutes 30% of total protein in the body and has a basic structure of triple helix. The collagen plays an essential role in providing a scaffold for cellular support, and thereby, affects cell attachment, migration, proliferation, differentiation, and survival.

In literature, there is no report that cell-containing collagen or fibrin gel was directly stimulated. Furthermore, there is no study that the physical force is loaded to a bottom of the collagen gel or fibrin gel periodically. According to an embodiment of the present invention, the bottom of gel is loaded with the physical forces in the form of “transverse impulse loading” to collagen gel by using the mechanical stimulator, which the present inventors designed. The present inventors found that synthesis of collagen and tensile strength had been dramatically increased by loading physical forces to the cell-containing collagen gel periodically, and then developed the present invention.

Because the “transverse impulse loading” was applied to the collagen gel from the below, the collagen gel moves upward and downward at each stimulation cycle. For example, stimulation is composed of hit and brief resting period. If each stimulation is composed of two hits which are achieved by rotating the mechanical stimulator having two cam connected to the cam-shaft, the collagen matrix moves upward and downward two times at each stimulation cycle and then brief resting period will follows. Because of characteristics of “transverse impulse loading”, collagen matrix will receive complex combination of forces in the 3 dimensional directions.

The part of the gel to be stimulated can be any point of the gel. Preferably, the gel is fixed at its edge to act as a fixed end, and at least a part including the central portion is stimulated so as to transmit the stimulus to whole the gel. In addition, if the collagen gel is large, it is preferable to stimulate at least two parts or more at the same or different time.

In accordance with one embodiment of the present invention, the periodic mechanical stimulation can be loaded by various methods. For example, the stimulation can be loaded by mechanical stimulator rotating at a speed of 0.01 to 500 rpm per 1 minute, and more preferably 50 to 100 rpm per 1 minute. If the stimulating frequency is excessively low, the stimulation does not affect the porosity and tensile strength of the collagen matrix. The excessively high frequency can change the shape of the collagen matrix.

The stimulation strength is 1.0×10⁻⁷ to 1.0×10-1 N/m², and more preferably 1.0×10⁻⁴ to 2.0×10⁻² N/m². If the stimulation strength is lower than 1.0×10⁻⁷ N/m², the stimulation is not sufficient for inducing the porosity and tensile strength. If the strength exceeds 1.0×10⁻¹ N/m², the excessive stimulation causes the collagen matrix to separated from the culture vessel.

Preferably, the collagens used in the present invention include Type I collagen, Type III, and Type IV collagen, and more preferably Type I collagen. In addition, fibrins also can be mixed in the present invention.

The cells of the present invention can be selected from the group consisting of fibroblast, dermal sheath cell, mesenchymal stem cell, vascular endothelial cell, endothelial progenitor cell (EPC), keratinocyte, melanocyte, hairy cell, Langerhans cell derived from blood, endothelial cell derived from blood, blood cell, macrophage, lymphocyte, adipocyte, sebaceous gland cell, cartilage cell, bone cell, osteoblast, and Merkel's cell derived from blood. Preferably, the cells are derived from young human. The cells include normal cells, genetically-modified cells, and malignant cells. The cells obtained from each tissue can be cultured according to the general method known in the art.

The cells are mixed with collagen solution to a concentration of 1×10³ to 1×10⁷ cells, more preferably 1×10⁵ to 1×10⁶ cells, and most preferably 3×10⁵ to 8×10⁵ cells per 1 ml of collagen solution. If the cells are less than 1×10³ cells in the collagen solution, the synthesis of matrix proteins are not sufficient. If the cell concentration is higher than 1×10⁷ cells, it may induce contraction of collagen matrix. The mixed collagen solution can be prepared in accordance with the methods known well to the art. Type I collagen can be extracted from tissues including rat-tails. To construct cell-embedded collagen gels, cultured cells were suspended in collagen solution, which was made by mixing eight volumes of type I collagen solution with one volume of 10× concentrated DMEM and one volume of neutralizing buffer.

In the embodiment of the present invention, fibroblast derived from the skin are mixed with collagen gel, and cultured in vitro in culture vessels which is made from elastic membrane. The material of the culture vessel is elastic and can be used for culturing animal cells. The shape of the culture vessel is not limited, but for example is circular, rectangular, plate-shaped, tube-shaped, and etc. The culture vessel is made from material selected from the group consisting of polyethylene, polypropylene, copolymer of polyethylene and polypropylene, silicone and a mixture thereof, but not limited thereto.

The culture method of collagen matrix according to the present invention is to increase the tensile strength of the collagen matrix by loading the periodic stimulation. In this invention, the stimulation method is simple and does not require the expensive machine and reagent. Thus, the culturing method of the present invention is cost-effective, and can produce the collagen matrix with high porosity and tensile strength.

Another embodiment of the present invention relates to a collagen matrix with increased porosity and tensile strength which are prepared according to the method as described above. The collagen matrix has a mean pore size of 0.1 to 100 μm, a porosity of 20% to 70%, and a tensile strength of 5 N/cm² to 200 N/cm², and more preferably 7 to 100 N/cm².

In order to measure porosity, usually the ratio of pore volume (water volume) to the total volume (dry material) was calculated after saturating the subject with water. However, due to the hydrophilic property of the collage matrix, the porosity is defined as percentage ratio of pore area to the total area, which are obtained from the two-dimensional electron-microscopic picture.

In the present invention, the tensile strength was measured in a saturation state with water, after removing remaining culture solution from the collagen matrix. Results showed that the tensile strength of the collagen matrix was increased by mechanical stimulation.

To understand the mechanism of these finding, expression levels of several molecules were examined. Expectedly, increased m-RNA levels of Type I procollagen and fibronectin were observed and MMP-1 levels were also increased. MMP-1 belonging to the membrane-bound MMPs, can digest extracellular matrix proteins such as procollagen Type I and fibronectin. MMP-1 activity is suppressed by tissue inhibitor of metalloproteinase-1 (TIMP-1) and TIMP-2. In this invention, increased levels of type I procollagen was observed. Thus, it can be said that increased expression of MMP-1, which were balanced by increased levels of TIMP-1 and -2, may remodel the collagen matrix.

Obtained collagen matrix with increased porosity and tensile strength, can be used for culturing the artificial skin or artificial organ. In an embodiment of the present invention, the epidermal cells, preferably keratinocytes are cultured on the collagen matrix. The collagen matrix can be a dermal substitute and provides the mechanical scaffold for the skin. Preferably, the dermal substitute of the present invention is the collagen matrix prepared by culturing it with above periodic mechanical stimulation.

Further embodiment of the present invention provides a method of preparing an artificial skin comprising the steps of:

a) preparing cell-containing collagen matrix according to the method of the present invention as described above;

b) inoculating and culturing keratinocytes at a concentration of 2×10⁴ to 2×10⁵ cells/cm² on the collagen matrix;

Step a) is performed according to the method of culturing the collagen matrix of the present invention as described above.

In addition, the present invention provides the artificial skin prepared according to the culturing method of the present invention. The artificial skin of the present invention has morphologic properties similar to the natural human skin.

The present invention provides the collagen matrix by stimulating cell-containing collagen and/or fibrin gels. By using mechanical stimulation, present invention can provide collagen matrix with good porosity and tensile strength which resemble those of human tissue.

The present invention is further explained in more detail with reference to the following examples. These examples, however, should not be interpreted as limiting the scope of the present invention in any manner

### Example 1

### Preparation of the Collagen Matrix with Increased Porosity and Tensile Strength

1-1: Manufacture of Mechanical Stimulator

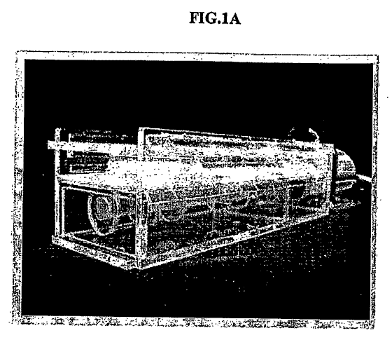

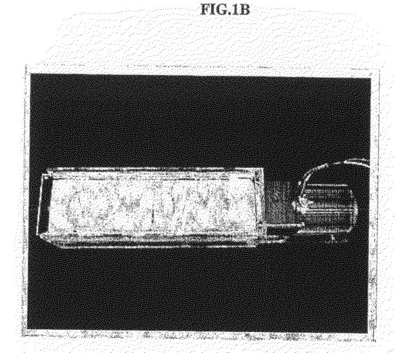

As shown in FIGS. 1A and 1B, the mechanical stimulator includes two oval-shaped cams connected to the cam-shaft which are located in position corresponding to the rubber plate and loads the force up to the rubber plate by contacting the rubber plate. Rubber plates are located on a upper part of a housing box and have the same size as the bottom of culture vessel. While the cam-shaft rotates, the two cams load the physical forces upward the central portion of rubber plate bottom on which the culture vessel presents.

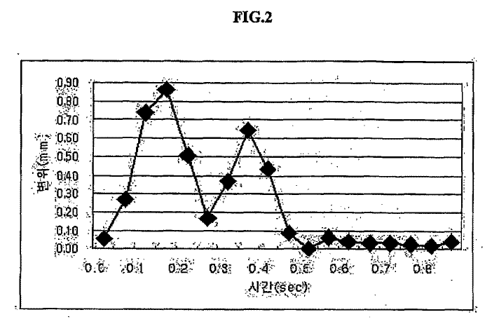

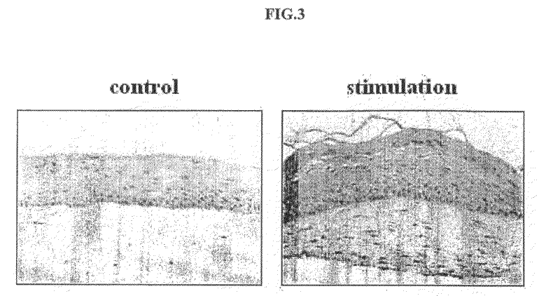

Thus, the collagen gel adhered to the culture vessel moves periodically. Because the culture vessel is fixed and adheres closely to the rubber plate, especially the edge of bottom of the culture vessel is fixed. The cycle of stimulation enforced on the bottom of the culture vessel was 72 rpm (0.8356 sec/cycle, 1.2 Hz). As shown in FIG. 2, the physical forces can be described as term “transverse impulse loading.”

In addition, the finite element analysis of the stimulation pattern was performed by using MSC.Nastran™ for Window 2003 (MSC Software Corporation, CA, USA) to test load which is given to the bottom of the culture vessel. As a result, the loaded maximum force was 1.7×10⁻³ N/m², the frequency of stimulation was 60 rpm per 1 minute.

1-2: Culturing the Cells and Cell-Containing Matrix

Human keratinocytes and dermal fibroblasts were isolated from human foreskins obtained during circumcision. Skin specimens were processed according to the method of Rheinwald and Green, as modified in our laboratory using thermolysin (Sigma Chemical Co., St. Louis, Mo.). Keratinocytes were cultured in keratinocyte growth medium (KGM, Clonetics, San Diego, Calif.), fibroblasts in Dulbecco's modified Eagle's medium (DMEM) supplemented with 10% fetal bovine serum (FBS). Type I collagen was extracted from rat tail tendons by stirring in 1/1000 glacial acetic acid at 4° C. for 48 h. Cell-containing collagen matrix were made by mixing eight volumes of type I collagen with one volume of 10× concentrated DMEM and one volume of neutralization buffer (0.05 N NaOH, 0.26 mM NaHCO₃, and 200 mM HEPES) and adding 5×10⁵ fibroblasts. Three milliliters of this mixture was then poured into a 30 mm polycarbonate filter chamber (3.0 cm Millicell; Millipore, Bedford, Mass.), and culture medium was added after gelation at 37° C. Then, cell-containing collagen matrix was stimulated using the method which was described in Example 1-1.

### Example 2

### Preparation of an Artificial Skin with Increased Tensile Strength

To reconstruct SEs, keratinocytes were inoculated and added with mixture of DMEM and Ham's F12 (3:1), supplemented with 5% FBS, 0.4 μg/ml hydrocortisone, 1 μM isoproterenol, 5 μg/ml insulin, 10 ng/ml epidermal growth factor (Invitrogen Co., Carlsbad, Calif.), 1 ng/ml bFGF (Sigma Chemical Co., St. Louis, USA), and 25 μg/ml ascorbic acid. SEs were submerged for a day, and then air-liquid exposed for an additional 12 days. After culturing, SEs were fixed to produce a paraffin block, and stained by hematoxylon-eosin.

### Experimental Example 1

### Dry Weight of Collagen Matrix

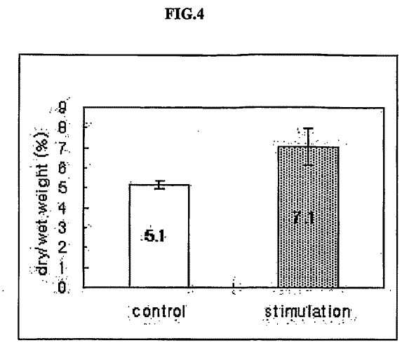

The dry weight of samples (1 cm×1 cm) was examined after freeze dry. The dry-weight was measured and calculated as a percentage of total wet weight (FIG. 4).

As shown in FIG. 4, percentage dry weight of the stimulated group was 7.1±0.9% [dry weight of 31.3±5.7 mg (26 at first time, 30.1 at second time, 37.3 mg at third time), total wet weight of 442.8±72.2 mg (360.4 at first time, 495.3 at second time, 472.6 mg at third time)]. The percentage dry weight of the un-stimulated group was 5.1±0.2% [dry weight of 28.8±4.6 mg (25.6 at first time, 26.4 at second time, 33.9 mg at third time), total wet weight of 559.5±114.4 mg (481.4 at first time, 506.2 at second time, 690.8 mg at third time)]. Results showed that percentage of dry weight increased by mechanical conditioning (from 5.1%±0.2% to 7.1%±00.9%).

### Experimental Example 2

### Analysis of Porosity

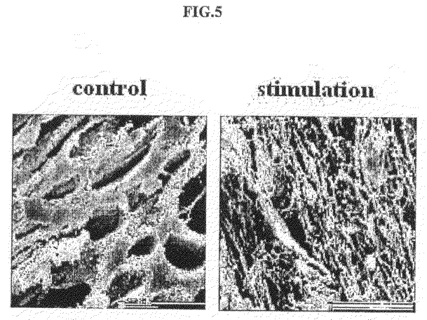

To investigate the effect of the mechanical simulation, the porosity of the collagen matrix was examined. Especially, the cross-section was observed using a scanning electron microscope (FIG. 5). In order to measure porosity, usually the ratio of pore volume (water volume) to the total volume (dry material) was calculated after saturating the subject with water. However, due to the hydrophilic property of the collage matrix, the porosity is newly defined as percentage ratio of pore area to the total area, which are obtained from the two-dimensional electron-microscopic picture.

The collagen matrix obtained in Example 1-2 had mean of 59.1±4.7% (62.9% at 1st, 58.7% at 2nd, 53.4% at 3rd, 64.5% at 4th, 55.9% at 5^{th} experiment) which was much higher than the porosity of control groups (mean of 34.3±3.0%, 29.8% at 1st, 35.6% at 2nd, 37.3% at 3rd, 36.1% at 4th, 32.8% at 5^{th} experiment). The pore size was various but generally compact in stimulated groups. As shown in FIG. 5, numerous bundles, which seemed to be newly synthesized, were observed in stimulated group and much smaller pores were observed compared to un-stimulated groups.

### Experimental Example 3

### Measurement of the Tensile Strength

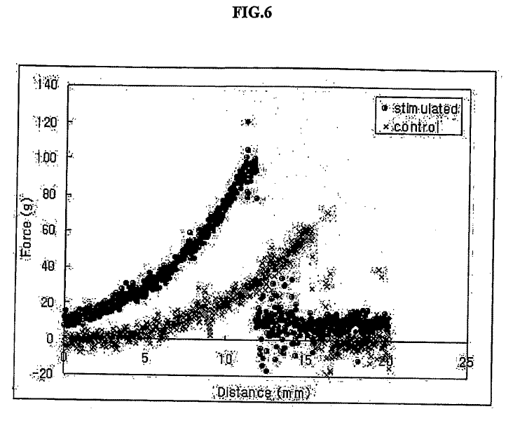

The tensile strength of the collagen matrix was measured by using Texture analyzer (TA-XT2i Texture Analyser, Stable Micro Systems, Godalming, UK). As shown in FIG. 6, collagen matrix from the stimulated group required more extension force than that of the unstimulated group.

In this experiment, the tensile strength was measured in a saturation state with water, after removing remaining culture solution from the collagen matrix. Tensile modulus of the control and stimulated group were 12.3±3.4 N/cm² (8.2 at 1st, 9.0 at 2nd, 15.7 at 3rd, 11.3 at 4th, 17.1 N/cm² at 5^{th} experiment), and 23.5±4.8 N/cm² (18.4 at 1st, 17.6 at 2nd, 28.1 at 3rd, 22.5 at 4th, 23.5 N/cm² at 5^{th} experiment), respectively. In every test, the tensile modulus of the stimulated group was about two times as that of the control group.

### Experimental Example 4

### Expression of Extracellular Matrix Proteins

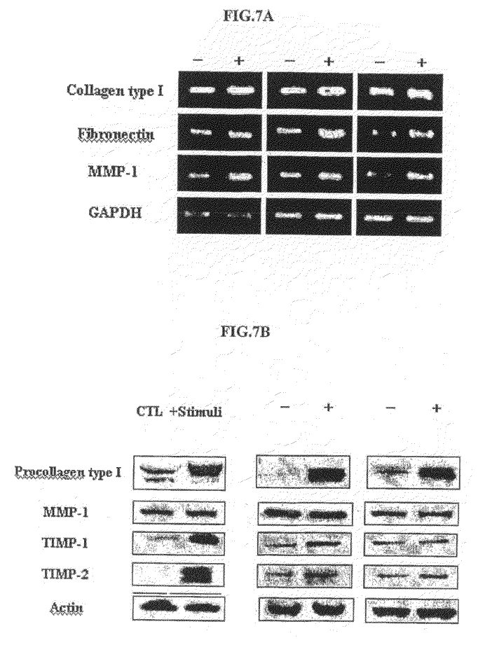

To investigate the effect of periodic stimulation on the collagen synthesis and expression of extracellular matrix proteins, 500 mg of collagen matrix obtained in Example 1-2, and 500 mg of control sample were prepared and RNA was also prepared by using TRIzol reagent (Cat. No. 15596-026, Gibco BRL/Invitrogen). Only a small amount of RNA were extracted from the control collagen gel which was obtained shortly after being solidified, but about 20 μg of RNA was extracted from the collagen matrix obtained from the Example 1-2. Extracted RNA was amplified by RT-PCR (reverse-transcription polymerase chain reaction) to show the result in FIG. 7A. More specifically, total RNA was isolated using TRIzol reagent (Gibco, Grand Island, N.Y.), and 2 ug of RNA were reverse transcribed using the reverse transcription system from Promega (Madison, Wis.) according to the manufacturer's instructions. The obtained cDNA was amplified using following primers:

* procollagen type I
forward primer: 5′-CTCGAGGTGGACACCACCCT-3′
reverse primer: 5′-CAGCTGGATGGCCACATCGG-3′
* MMP-1
forward primer: 5′-ATTCTACTGATATCGGGGCTTTGA-3′
reverse primer: 5′-ATGTCCTTGGGGTATCCGTGTAG-3′
* fibronectin
forward primer: 5′-AGGTTCGGGAAGAGGTTGTT-3′
reverse primer: 5′-TGGCACCGAGATATTCCTTC-3′.

The PCR products were visualized by electrophoresis on 1.5% agarose gels and ethidium bromide staining. The obtained cDNA was also amplified by using specific primers for GAPDH as follows:

Forward primer: 5′-CCACCCATGGCAAATTCCATGGCA-3′
Reverse primer: 5′-TCTAGACGGCAGGTCAGGTCCACC-3′.

FIG. 7A showed that m-RNA levels of procollagen Type I and fibronectin were increased in the stimulated group compared to the control group.

The protein expression level was analyzed by western blotting method. Cultured dermal substitutes were lysed in buffer [62.5 mM Tris-HCl (pH 6.8), 2% SDS, 5% β-mercaptoethanol, 2 mM phenylmethylsulfonyl fluoride, protease inhibitors (CompleteTM, Roche, Mannheim, Germany), 1 mM Na3VO4, 50 mM NaF, and 10 mM EDTA]. Twenty micrograms of protein per lane was separated by SDS-polyacrylamide gel electrophoresis and blotted onto nitrocellulose membranes, saturated with 5% dried milk in Tris-buffered saline containing 0.4% Tween 20. Blots were incubated with the appropriate primary antibodies at a dilution of 1:1000, and then further incubated with horseradish peroxidase-conjugated secondary antibody. Bound antibodies were detected using an enhanced chemiluminescence plus kit (Amersham International, Little Chalfont, U.K.). The following antibodies were used: monoclonal mouse antibodies to Procollagen type I (SP1.D8, provided by Dr. Heinz Furthmayr, Yale University School of Medicine), MMP1 (Oncogene, IM35L, La Jolla, Calif.), TIMP1 (Santa Cruz Biotechnology, Inc., sc-6832, Santa Cruz, CA), TIMP2 (Santa Cruz Biotechnology, Inc., sc-21735), and goat antibody to Actin (Santa Cruz Biotechnology, Inc., sc-1616).

As a result, it is found that physical stimulation dramatically increased the levels of procollagen type I protein compared to control samples. Furthermore, the levels of TIMP-1 and TIMP-2, which suppress MMP-1 activity, increased compared to control samples. These findings explain the levels of MMP-1 are similar in both groups although the mRNA levels of MMP-1 are higher in stimulated group. Thus, it can be said that increased production of procollagen Type I protein and controlled digestion by MMP-1 protein produced collagen matrix with more fine pores and increased tensile strength.

While the present invention has been described in detail with reference to the preferred embodiments, those skilled in the art will appreciate that various modifications and substitutions can be made thereto without departing from the spirit and scope of the present invention as set forth in the appended claims.

1-19. (canceled)

20. A method for producing a cell-containing collagen matrix, which comprises the steps of:
a) preparing a gel by mixing mammalian cells and a solution containing a material selected from the group consisting of collagen, fibrin and a mixture thereof; and
b) stimulating the gel by applying one or more periodical and discontinuous mechanical impulses to said gel in order to immobilizing said mammalian cells on said material.

21. The method of claim 20, wherein the mechanical impulses are applied to a bottom of a culture vessel which contains said gel in order to increase generation of collagen by the mammalian cells.

22. The method of claim 20, wherein the collagen is at least one selected from the group consisting of Type I collagen, Type III collagen and Type IV collagen.

23. The method of claim 20, wherein the mechanical impulses are transverse impulses applied to the bottom plane of the culture vessel which contains said gel.

24. The method of claim 20, wherein the collagen matrix has pores with a diameter of 0.1 μm to 100 μm, porosity of 10% to 90%, and tensile strength of 1 N/cm² to 200 N/cm².

25. The method of claim 20, wherein the strength of the mechanical impulses is 1.0×10⁻⁷ N/m² to 1.0×10⁻¹ N/m².

26. The method of claim 20, wherein the frequency of the mechanical impulses is 0.01 cycles/min to 500 cycles/min.

27. The method of claim 20, wherein the step b) is performed by applying the mechanical impulses to said culture vessel made of elastic material.

28. The method of claim 20, wherein said elastic material is selected from the group consisting of polyethylene, polypropylene, ethylene-propylene copolymer and silicon.

29. The method of claim 28, wherein the gel is fixed peripheral side of the culture vessel and then is applied to mechanical impulses at least one site of the bottom of the culture vessel including the central site thereof.

30. The method of claim 20, wherein the stimulation is applied at least two sites of the bottom of the collagen gel independently.

31. The method of claim 20, wherein the mechanical impulses are generated by at least one cam attached to the cam-shaft.

32. The method of claim 31, wherein the cam-shaft rotates at a speed of 0.01 rpm to 500 rpm.

33. The method of claim 20, wherein the mammalian cells are mixed at a concentration of 1×10³ cells/ml of the solution to 1×10⁷ cells/ml of the solution.

34. The method of claim 20, wherein the mammalian cell is selected from the group consisting of fibroblast, dermal sheath cell, dermal papilla cell, mesenchymal stem cell, embryonic stem cell, endothelial cell, endothelial progenitor cell (EPC), outer root sheath cell, keratinocyte, melanocyte, hair cell, Langerhans cell derived from blood, endothelial cell derived from blood, blood cell, macrophage, lymphocyte, adipocyte, sebaceous gland cell, cartilage cell, bone cell, osteoblast, and Merkel's cell derived from blood.

35. A collagen matrix having pores with a diameter of 0.1 to 100 μm, a porosity of 10% to 90%, and a tensile strength of 1 N/cm² to 200 N/cm².

36. A collagen matrix having pores with a diameter of 0.1 to 100 μm, a porosity of 10% to 90%, and a tensile strength of 1 N/cm² to 200 N/cm², wherein the collagen matrix is the collagen matrix including a mammalian cell being prepared according to the method of claim 20.

37. A method for culturing of artificial skin or organs, which comprises a collagen scaffold comprising the collagen matrix being prepared according to the method of claim 20.

38. A method of preparing a collagen matrix according to claim 20, wherein said collagen matrix is incorporated in a therapeutic filler or an esthetic filler.
